(19) United States
(12) Patent Application Publication
(10) Pub. No.: US 2015/0037393 A1
(43) Pub. Date: Feb. 5, 2015

(54) ADHESIVE PATCHES FOR USE IN SUNLESS TANNING

(71) Applicants: Peter Robert Millman, St. Louis, MO (US); Sydney Skae Millman, St. Louis, MO (US)

(72) Inventors: Peter Robert Millman, St. Louis, MO (US); Sydney Skae Millman, St. Louis, MO (US)

(21) Appl. No.: 13/970,851

(22) Filed: Aug. 20, 2013

Related U.S. Application Data
(60) Provisional application No. 61/860,350, filed on Jul. 31, 2013.

(51) Int. Cl. C09J 7/02; A61Q 19/04; A61K 9/70

(52) U.S. Cl. CPC C09J 7/02; A61K 9/70; A61Q 19/04
USPC 424/448; 428/337; 442/149; 428/317.3; 424/59

(57) ABSTRACT

Adhesive patches for protecting skin imperfections such as age spots, from discoloring and/or darkening during sunless tanning are provided herein. The adhesive patches are sized and shaped to fully or partially cover the skin imperfections during application of a sunless tanning product such to provide a user with uniform color development over the entire skin, and, in some embodiments, to allow for the skin imperfection to appear less visible.

### CROSS-REFERENCE TO RELATED APPLICATIONS

This application claims priority from U.S. Provisional Patent Application Ser. No. 61/860,350, filed on Jul. 31, 2013, which is herein incorporated by reference in its entirety.

### BACKGROUND OF THE DISCLOSURE

The field of the disclosure relates generally to protecting skin imperfections during sunless tanning. More particularly, adhesive patches for protecting skin imperfections such as age spots, wounds, scars, and acne from discoloring and/or darkening during sunless tanning, and in particular, spray tanning, are provided herein.

The sun's rays are known to produce ultraviolet radiation, such as sunburn that can have detrimental effects on the skin. Excessive exposure can lead to skin wrinkling, age spots, and even skin cancer.

As people become more aware of the harmful effects of the sun, products such as sunless tanning products are gaining popularity. These products typically employ a sunless tanning agent, such as dihydroxyacetone (DHA), to impart color onto the skin that provides the impression of a tan produced by exposure to the sun. More particularly, DHA gives skin a brownish color by reacting with amino acids present in the sebum and stratum corneum by the Maillard reaction.

However, these products have drawbacks that make their use inconvenient and undesirable to many consumers who use such products. One problem is that the distribution and nature of the amino acids is not uniform on the surface of the skin and, because of that, the intensity and shade of the color obtained may vary from one place to another on the treated skin, thereby causing the skin to have an unnatural look. Further, skin imperfections such as age spots, acne, and scars typically vary in color and darkness from healthy, imperfection-free skin, and thus, when tanning products are applied to skin imperfections, these areas appear darker than the surrounding skin.

Based on the foregoing, there is a need for a product that overcomes these problems by protecting areas of skin having imperfections from the negative effects of sunless tanning products. More particularly, it would be advantageous if a user could partially or fully protect a skin imperfection from a sunless tanning product such to provide uniform color development across the entire skin area. It would be further advantageous if the user could control the exposure of the skin imperfection to the sunless tanning product such that, after application of the product, the user's skin appears both uniformly tanned and the skin imperfection is less visible.

### BRIEF DESCRIPTION OF THE DISCLOSURE

The present disclosure is directed to adhesive patches for protecting skin imperfections such as age spots, wounds, scars, and acne from discoloring during sunless tanning, and in particular, spray tanning. The adhesive patches are sized and shaped to fully or partially cover the skin imperfections during application of a sunless tanning product such to provide a user with uniform color development over the entire skin, and, in some embodiments, to allow for the skin imperfection to appear less visible.

Accordingly, in one embodiment, the present disclosure is directed to an adhesive patch for protecting skin imperfections during sunless tanning. The adhesive patch comprises: a substrate and an adhesive composition on at least a first face of the substrate. The substrate has a width along its greatest extent of from about 1/16″ to about 7/16″.

In another embodiment, the present disclosure is directed to a strip comprising a plurality of adhesive patches for protecting skin imperfections during sunless tanning. The plurality of adhesive patches comprises at least a first adhesive patch and a second adhesive patch, the first adhesive patch and the second adhesive patch independently comprising a substrate and an adhesive composition on at least a first face of the substrate. The first adhesive patch and the second adhesive patch independently have a width along their greatest extent of from about 1/16″ to about 7/16″, and wherein the width along its greatest extent of the first adhesive patch is greater than the width along its greatest extent of the second adhesive patch.

In yet another embodiment, the present disclosure is directed to a method of protecting skin imperfections during sunless tanning. The method comprises: identifying a skin imperfection on the skin of a user; applying an adhesive patch to the skin imperfection; applying a sunless tanning product; and removing the adhesive patch from the skin imperfection.

### BRIEF DESCRIPTION OF THE DRAWINGS

FIG. 1 depicts one embodiment of a strip including a plurality of adhesive patches for protecting skin imperfections according to the present disclosure.

FIG. 2A depicts the body-facing surface of an adhesive patch for protecting skin imperfections according to one embodiment of the present disclosure.

FIG. 2B depicts the outer facing surface of an adhesive patch for protecting skin imperfections according to one embodiment of the present disclosure.

FIG. 3 depicts the outer facing surface of a substrate of an adhesive patch in one embodiment according to the present disclosure.

FIG. 4 depicts one embodiment of a strip including a plurality of adhesive patches adhered to a peel sheet according to the present disclosure.

FIG. 5 depicts one embodiment of a peel sheet adhered to the body-facing surface of a substrate of an adhesive patch according to the present disclosure.

### DEFINITIONS

It should be noted that, when employed in the present disclosure, “sunless tanning product” refers to a product including a self-tanning agent for imparting color onto a user's skin without the use of sun to provide an impression of a tan produced conventionally by exposure to the sun. Self-tanning agents are known in the art, and include, for example, dihydroxyacetone (DHA), melanin, mahakanni (eclipta alba), erythrulose, or any mixtures thereof. Most commonly used is DHA, which is an aliphatic ketone capable of coloring the skin. The reaction between DHA and the proteins in the skin leads to the appearance of tanned skin. Typically, the reaction requires at least 2 hours. The sunless tanning product may further include an application indicator agent to act as a visual aid during application for allowing the user to apply the sunless tanning product evenly and uniformly and a color fixative to facilitate and/or enhance color retention on the skin's surface. The sunless tanning product may be in the form of a lotion, gel, cream, aerosol spray, non-aerosol spray, sol, and combinations thereof.

As used herein, “skin imperfection” refers to any type of skin blemish or discoloration or area of the skin that differs in shape or texture from the user's normal healthy skin. Common skin imperfections include, for example, age spots, freckles, acne, wounds, scars, birthmarks, chloasma patches, actinic keratosis, actinic lentigines, rashes, hives, and the like. In particular embodiments, the skin imperfection protected by the adhesive patch of the present disclosure is one or more of an age spot, acne, wound, or a scar.

As used herein, the term “polymer” generally includes, but is not limited to, homopolymers, copolymers, such as block, graft, random and alternating copolymers, terpolymers, etc., and blends and modifications thereof. Furthermore, unless otherwise specifically limited, the term “polymer” shall include all possible geometrical configurations of the material. These configurations include, but are not limited to, isotactic, syndiotactic and random symmetries.

As used herein, “body-facing surface” means that face of the substrate which is intended to be disposed toward or placed adjacent to the body of a user during ordinary use. The “outer-facing surface” means that face that is on the opposite side of the substrate from the body-facing surface. The outer-facing surface is an outward face of the substrate and is intended to be disposed to face away from the user's body during ordinary use.

### DETAILED DESCRIPTION OF THE DISCLOSURE

### Adhesive Patches

Adhesive patches for protecting skin imperfections such as age spots, wounds, scars, and acne from discoloring during sunless tanning, and in particular, sunless spray tanning, are provided herein. The adhesive patches of the present disclosure provide a substrate which is designed to adhere to the skin of a user in the area which may need protection from application of a sunless tanning product, typically one or more of an age spot, wound, scar, acne, and the like. The patches are typically sized and shaped to the skin imperfections to which the patches are to be applied.

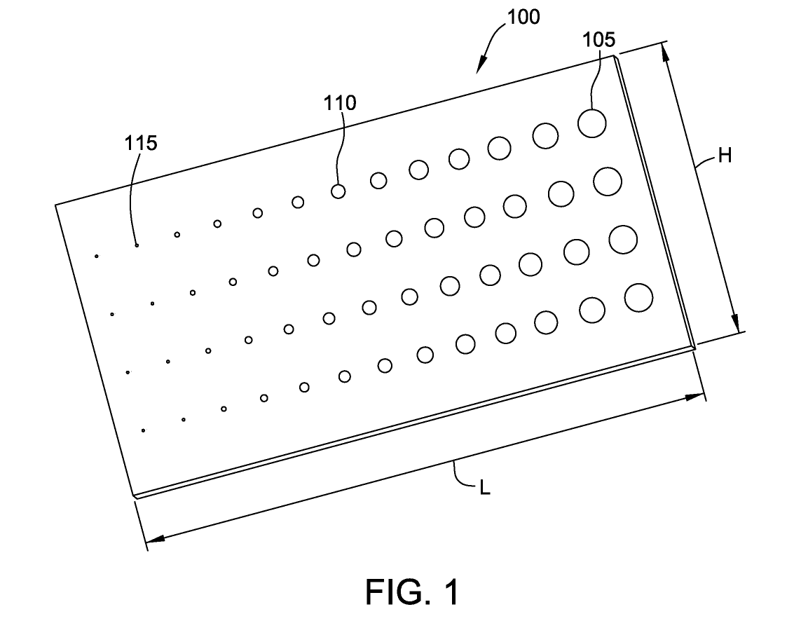

To gain a better understanding of the present disclosure, attention is directed to the Figures of the present specification. As is shown in FIG. 1, a strip 100 has a longitudinal direction L and a lateral direction H. The strip 100 includes a plurality of adhesive patches (e.g., 105, 110, 115) for application to a user's skin. As shown as three separate sized adhesive patches 105, 110, 115, it should be understood that strip 100 can include more or less sizes of adhesive patches without departing from the present disclosure. That is, strip 100 can include adhesive patches having two different sizes, four different sizes, five different sizes, and even more than five different sizes.

Adhesive patches 105, 110, 115 are arranged in columns and rows. It should be understood by one skilled in the art, however, that the adhesive patches can be arranged other than as shown, such as in a single column, in a single row, or combinations thereof. Further, while shown in FIG. 1 as a strip 100 having four columns and twelve rows, it should be understood by one skilled in the art that the strip can include more or less columns and/or more or less rows without departing from the present disclosure. For example, in one particularly preferred embodiment, the strip includes four columns and ten rows. In should be further understood by one skilled in the art that as adhesive patches 105, 110, 115 are of different sizes, strip 100 may alternatively include more or less columns and/or rows in a first portion as compared to a second portion of strip 100, and further as compared to a third portion of strip 100 and so on. By way of example, on one strip, there may be four columns and four rows of adhesive patches 105, three columns and four rows of adhesive patches 110, and two columns and four rows of adhesive patches 115. In another example, on one strip, there may be four columns and four rows of adhesive patches 105, four columns and three rows of adhesive patches 110, and four columns and two rows of adhesive patches 115.

Adhesive patches 105, 110, 115 may vary in size and/or shape such that the patches 105, 110, 115 can easily and effectively cover the skin imperfection to which they are to be applied. More particularly, adhesive patches 105, 110, 115 may be sized and configured to fit precisely over a skin imperfection, thus allowing protection of the skin imperfection from the sunless tanning product while allowing the surrounding skin exposure to the product.

As shown in FIG. 1, adhesive patch 105 has a greater diameter than adhesive patch 110, and adhesive patch 110 has a greater diameter than adhesive patch 115. As should be understood in the art, while shown as circular adhesive patches, the adhesive patches can have a variety of shapes, such as rectangles, squares, ovals, and the like, without departing from the present disclosure. It should be further understood that when configured in any of the above-described shapes, adhesive patch 105 may have a width along its greatest extent greater than the width of adhesive patch 110 along its greatest extent, and further, adhesive patch 110 may have a width along its greatest extent greater than the width of adhesive patch 115 along its greatest extent.

Typically, adhesive patches 105, 110, 115 independently have widths along their greatest extents that range from about 1/16″ to about 7/16″, including from about 2/16″ to about 5/16″, and including from about 2/16″ to about 3/16″. In embodiments where the skin imperfection is larger than adhesive patches 105, 110, 115, it should be understood that the patches can be combined and/or clustered into a size and shape that effectively covers the skin imperfection.

Adhesive patches 105, 110, 115 can be of any color known in the art, and in some embodiments, may vary in color on a single strip 100. In one embodiment, adhesive patches 105, 110, 115 are skin toned. As used herein, “skin toned” refers to the color of the user's skin.

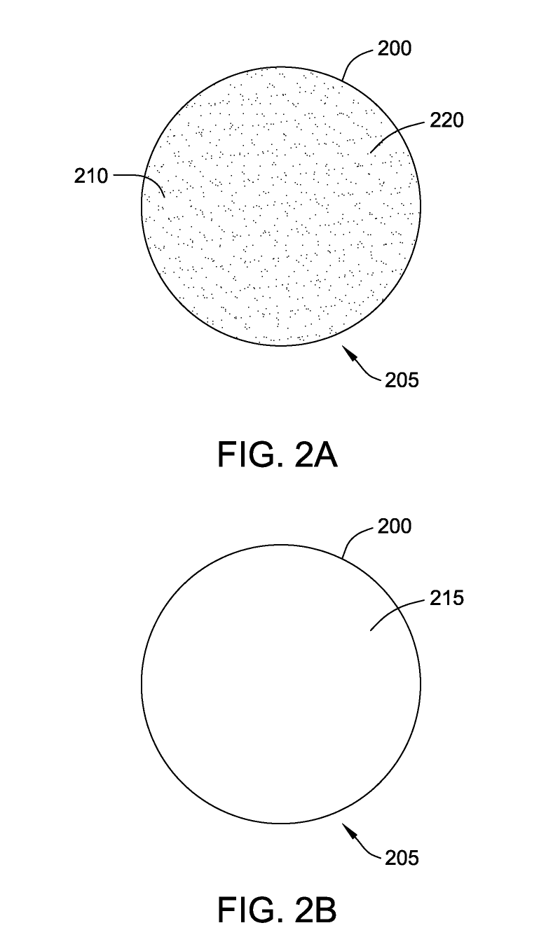

Shown in FIGS. 2A and 2B is an exemplary adhesive patch 205 for use with the strip 100 of FIG. 1. Adhesive patch 205 includes a substrate 200 having a first face 210 and a second face 215. Substrate 200 can be made from any suitable material known in the art, including, for example, woven materials, nonwoven materials, composite materials, polymeric films, film-nonwoven fabric laminates, and combinations thereof. Examples of a nonwoven fabric useable in the substrate 200 include, for example, an airlaid nonwoven web, a spunbond nonwoven web, a meltblown nonwoven web, a bonded-carded web, a hydroentangled nonwoven web, a spunlace web or the like, as well as combinations thereof. Other examples of suitable materials for constructing the substrate 200 can include rayon, bonded-carded webs of polyester, polypropylene, polyethylene, nylon, or other heat-bondable fibers, finely perforated film webs, net-like materials, and the like, as well as combinations thereof. These webs can be prepared from polymeric materials such as, for example, polyolefins, such as polypropylene and polyethylene and copolymers thereof, polyesters in general including aliphatic esters such as polylactic acid, nylon or any other heat-bondable materials.

In some embodiments, it may be desirable to the user to allow the skin imperfection some exposure to the sunless tanning product, however, in an amount less than that of the surrounding skin. In such embodiments, it may be desirable for adhesive patch 205 to include a porous substrate. For example, when the substrate is a film or a film laminate, the film may be apertured or otherwise be made porous to allow the user to control the amount of exposure the skin underneath adhesive patch 205 has to the sunless tanning product. Alternatively, a plurality of pores, perforations, apertures or other openings, as well as combinations thereof, can be made in nonwoven and woven materials for use as substrate 200 to allow the skin imperfection some exposure to the sunless tanning product.

Other examples of suitable materials for the substrate 200 are composite materials of a polymer and a nonwoven fabric material. The composite materials are typically in the form of integral sheets generally formed by the extrusion of a polymer onto a nonwoven web, such as a spunbond material.

First face 210, also referred to herein as the “body-facing surface”, typically includes a pressure sensitive adhesive composition 220 to removably couple the substrate to the skin of a user. The adhesive composition 220 may cover the entire first face 210 of the substrate 200 (as shown in FIG. 2A). Alternatively, the adhesive composition 220 may be placed on a portion of the first face (not shown in the drawings). The adhesive composition 220 may also be placed in a pattern on the first face 210 of the substrate 200. The adhesive composition 220 can be applied to the first face 210 of the substrate 200 using any known process including inkjet printing, screen printing or extruding the adhesive composition from one or more nozzles, slot coating and the like.

The adhesive composition 220 may be positioned on the first face 210 of the substrate 200 in an open pattern or a closed pattern. By “open pattern” is meant that the adhesive composition can have an intermittent or discontinuous pattern which does not substantially cover the first face. For example, there may be breaks in the adhesive composition at certain portions of the first face 210. “Closed pattern” means the adhesive composition 220 would cover the entire first face of the substrate.

In one embodiment, the pattern of the adhesive composition 220 will substantially encircle the outer edge of first face 210. An example of an “open” pattern of the adhesive composition would be to have individual beads of adhesive composition applied in a discontinuous fashion.

The adhesive composition may be applied in a pattern of small discrete dots so as to leave numerous areas free from adhesive. Alternatively, the adhesive composition may be applied as a continuous bead, or may be applied as a series of semi-continuous beads. Other suitable adhesive patterns may be selected for applying the adhesive 220 to the first face 210 of the substrate 200. For example, adhesive patterns can be oval, swirls, various linear or non-linear arrays of adhesive longitudinally, and/or transversely oriented and reticulated webs having unobstructed interstices between the adhesive fibers or combinations thereof. As stated above, the adhesive patterns may be open or closed. The weights of adhesive compositions are limited to less than about 800 g/m², and generally less than about 400 g/m². Generally, the weight of the adhesive is at least 20 g/m². Typically, the adhesive composition is applied in an amount of from about 100 to about 400 g/m². The limitations on the basis weight of the adhesive composition are important to provide the correct adhesive characteristics for applying directly to the skin of the user. If the basis weight is too high, the adhesive patch will have a sticky feeling or otherwise uncomfortable feeling. If the basis weight of the adhesive composition is too low, there may be insufficient adhesion to the skin of the user.

Generally, the adhesive composition 220 is applied in a manner which is symmetrical about the longitudinal axis which bisects the substrate 200 and divides the substrate 200 into substantially equal portions. This symmetrical pattern provides the user a balanced feel when wearing the adhesive patch 205. The symmetrical pattern also reduces the perception of any associated discomfort when the adhesive patch 205 is removed from the skin.

Generally, any pressure sensitive adhesive composition known to those skilled in the art may be used, provided that the pressure sensitive adhesive composition is not a known irritant to human skin or that the adhesive composition is so aggressive that it causes pain to the user when the adhesive patch is removed from the skin. It is also desirable that the adhesive composition is selected such that the adhesive composition does not leave a substantial amount of an adhesive residue on the surface of the skin of the user when the adhesive patch is removed by the user after use. Particularly suitable pressure sensitive adhesive materials are disclosed in U.S. Pat. No. 6,213,993 to Zacharias et al., U.S. Pat. No. 6,620,143 to Zacharias et al., the disclosure of each is incorporated herein by reference and made a part hereof to the extent it is consistent herewith. Other suitable adhesives are disclosed in U.S. Pat. No. 5,618,281 to Batrabet et al., the disclosure of which is incorporated herein by reference and made a part hereof to the extent it is consistent herewith. Other known body adhesives, such as those described in U.S. Pat. No. 6,316,524 to Corzani et al. which is hereby incorporated to the extent it is consistent herewith, may also be used. Additional examples of pressure sensitive adhesive compositions include hydrogels, hydrocolloids, acrylics based adhesives, and rubber based adhesives, such as Kraton based adhesives.

It should be understood that the adhesive composition on the first face should form a sufficiently strong bond with the skin imperfection such as to prevent premature peeling, yet the composition needs to remain gently attached to the skin's surface so as to be easily and comfortably removed and/or reapplied. Accordingly, the adhesive composition 220 should provide a peel strength of composition to skin of from about 5 grams per inch to about 100 grams per inch and in another embodiment from about 20 grams per inch to about 100 grams per inch. In some embodiments, the adhesive composition has a peel strength of composition to skin of from about 75 grams per inch to about 500 grams per inch or even from about 100 grams per inch to about 200 grams per inch. To measure peel strength of the adhesive composition, a method similar to STM 5599 may be used. STM 5599 is fully described in U.S. application Ser. No. 12/267,806, which is incorporated herein for all relevant and consistent purposes.

In accordance with the present disclosure, skin benefit agents may suitably be included in the adhesive composition 220, the first 210 and combinations thereof. A skin benefit agent generally provides a skin benefit (e.g., functional, aesthetic, or heath benefit) to the user. For example, a skin benefit agent such as an antiperspirant can be beneficial to the adhesive composition as it will prevent the adhesive composition from weakening as a result of increased sweating in specific areas of the body for use with the adhesive patch.

Exemplary skin benefit agents may include for example: antiperspirants, deodorants, skin moisturizers, humectants, pH modulators, soothing agents, antimicrobials, preservatives, film formers, vitamins, and combinations thereof. Antiperspirant agents are those active ingredients generally found in antiperspirant products. In the Final Rule for U.S. Antiperspirant Drug Products for Over-the Counter Human Use; Final Monograph (68 Federal Register 34273-34293, Jun. 9, 2003) an “antiperspirant” refers to a drug product applied topically that reduces the production of perspiration (sweat) at that site. See 21 CFR 350.3 for definition and 21 CFR 350.10 for a listing of the U.S. Antiperspirant active ingredients. The following is a list of ingredients currently listed in the INCI Dictionary under this category: adipic acid/neopentyl glycol crosspolymer, aluminum chloride, aluminum chlorohydrate, aluminum chlorohydrex PEG, aluminum chlorohydrex PG, aluminum dichlorohydrate, aluminum dichlorohydrex PEG, aluminum dichlorohydrex PG, aluminum sesquichlorohydrate, aluminum sesquichlorohydrex PEG, aluminum sesquichlorohydrex PG, aluminum sulfate (aluminum sulfate buffered), aluminum zirconium octachlorohydrate, aluminum zirconium octachlorohydrex GLY, aluminum zirconium pentachlorohydrate, aluminum zirconium pentachlorohydrex GLY, aluminum zirconium tetrachlorohydrate, aluminum zirconium tetrachlorohydrex GLY, aluminum zirconium trichlorohydrate, aluminum zirconium trichlorohydrex GLY, Bursera Graveolens fruit oil, ferric chloride, Humulus Lupulus (Hops) cone extract, Hypericum Perforatum flower/twig extract, zirconium powder, and combinations thereof. Particularly preferred antiperspirants for use with the adhesive patches of the present disclosure include commercially available REACH Aluminum-Zirconium Complex (AZP) 908, and REACH 103, which is a chlorohydrate, both of which are commercially available from Reheis, Inc., Berkeley Heights, N.J.

Deodorants are agents that reduce or eliminate unpleasant odors and protect against the formation of malodor on bodily surfaces. Absorbents can act as deodorants if they have the ability to absorb malodorous chemicals. Also, chemical reactions can be used to destroy the malodorous substance in selected cases. Perfumes and the like can be used to mask the perception of malodor by the process of reodorization. Unpleasant odors also may be the result of microbiological activity. Thus, cosmetic biocides are ingredients frequently used in skin-surface deodorants. The following listing of deodorants is limited to those ingredients commonly used for this purpose: adipic acid/neopentyl glycol crosspolymer, Alpinia Uraiensis stalk/leaf water, aluminum chloride, aluminum chlorohydrate, aluminum chlorohydrex, aluminum chlorohydrex PEG, aluminum chlorohydrex PG, aluminum dichlorohydrate, aluminum dichlorohydrex PEG, aluminum dichlorohydrex PG, aluminum lactate, aluminum phenolsulfonate, aluminum sesquichlorohydrate, aluminum sesquichlorohydrex PEG, aluminum sesquichlorohydrex PG, aluminum sulfate, aluminum triphosphate, aluminum zinc oxide, aluminum zirconium octachlorohydrate, aluminum zirconium octachlorohydrex GLY, aluminum zirconium pentachlorohydrate, aluminum zirconium pentachlorohydrex GLY, aluminum zirconium tetrachlorohydrate, aluminum zirconium tetrachlorohydrex GLY, aluminum zirconium tetrachlorohydrex PEG, aluminum zirconium tetrachlorohydrex PG, aluminum zirconium trichlorohydrate, aluminum zirconium trichlorohydrex GLY, amber powder, ammonium phenolsulfonate, ammonium silver zinc aluminum silicate, benzalkonium bromide, benzalkonium cetyl phosphate, benzalkonium chloride, benzalkonium saccharinate, benzethonium chloride, Boesenbergia Pandurata Rhizome extract, bromochlorophene, t-Butyl methylphenoxy phenol, calcium magnesium silicate, Callicarpa Macrophylla flower extract, Candida Bombicola/glucose/methyl rapeseedate ferment, capryloyl gold of pleasure amino acids, cetylpyridinium chloride, chlorophyllin-copper complex, chlorothymol, chloroxylenol, Citrus Reticulata (Tangerine) peel oil, cloflucarban, colloidal platinum, Cuminum Cyminum seed extract, Curcuma Heyneana root powder, cyclopentadecanone, dequalinium chloride, dichlorophene, dichloro-m-xylenol, dimethicone/PEG-15 crosspolymer, dipotassium capryloyl glutamate, disodium capryloyl glutamate, disodium dihydroxyethyl sulfosuccinylundecylenate, domiphen bromide, ethylhexylglycerin, fermented vegetable, hexachlorophene, hydrolyzed Sasa Veitchii extract, ketoglutaric acid, lauryl isoquinolinium bromide, laurylpyridinium chloride, magnesium/aluminum/zinc/hydroxide/carbonate, mentha aquatica water, methylbenzethonium chloride, methyl undecylenate, Michelia Champaca flower oil, micrococcus/hydrolyzed nonfat milk ferment, octadecenedioic acid, oligopeptide-10, Pandanus Amaryllifolius leaf extract, Pelargonium Graveolens water, phenol, Phyllostachys Edulis stem extract, Piper Bede leaf oil, polyaminopropyl biguanide stearate, potassium capryloyl glutamate, Rosmarinus Officinalis (Rosemary) flower extract, saccharomyces/persimmon fruit juice ferment extract, Saccharomyces/Rhodobacter/Lactobacillus/Leuconostoc/Streptomyces/Griseus/Aspergillus/Bacillus ferment filtrate, Sasa Senanensis leaf extract, Sasa Senanensis leaf powder, silver copper zeolite, sodium bicarbonate, sodium capryloyl glutamate, sodium phenolsulfonate, sodium silver aluminum silicate, Stemmacantha Carthamoides root extract, totarol, triclocarban, triclosan, tricyclodecenyl propionate, Urginea Maritima tuber extract, zeolite, zinc lactate, zinc phenolsulfonate, zinc ricinoleate, zinc silicate, and combinations thereof.

Humectants are hydroscopic agents that are widely used as skin moisturizers. Their function is to prevent the loss of moisture from the skin and to attract moisture from the environment. Common humectants include, for example, glycerin, butylene glycol, betaine, sodium hyaluronate, and the like, and combinations thereof.

Soothing agents, also referred to as emollients, lubricate, sooth, and soften the skin surface. Exemplary emollients include oily or waxy ingredients such as esters, ethers, fatty alcohols, hydrocarbons, silicones, and the like, and combinations thereof.

Film formers, also referred to as skin barrier enhancers or occlusive materials, increase the water content of the skin by blocking water evaporation. These materials generally include lipids which tend to remain on the skin surface or hydrocarbons such as petrolatum and wax.

Rheology enhancers may help increase the melt point viscosity of the adhesive composition so that the composition readily remains on the surface of the substrate and does not substantially migrate into the interior of the substrate, while substantially not affecting the transfer of the composition to the skin. The rheology enhancers help the composition to maintain a high viscosity at elevated temperatures, such as those encountered during storage and transportation. Additionally, rheology enhancers can influence the overall consistency and skin feel of the composition.

Suitable rheology enhancers include combinations of alpha-olefins and styrene alone or in combination with mineral oil or petrolatum, combinations of di-functional α-olefins and styrene alone or in combination with mineral oil or petrolatum, combinations of alpha-olefins and isobutene alone or in combination with mineral oil or petrolatum, ethylene/propylene/styrene copolymers alone or in combination with mineral oil or petrolatum, butylene/ethylene/styrene copolymers alone or in combination with mineral oil or petrolatum, ethylene/vinyl acetate copolymers, polyethylene polyisobutylenes, polyisobutenes, polyisobutylene, dextrin palmitate, dextrin palmitate ethylhexanoate, stearoyl inulin, stearalkonium bentonite, distearadimonium hectorite, and stearalkonium hectorite, styrene/butadiene/styrene copolymers, styrene/isoprene/styrene copolymers, styrene-ethylene/butylene-styrene copolymers, styrene-ethylene/propylene-styrene copolymers, (styrene-butadiene)n polymers, (styrene-isoprene)n polymers, styrene-butadiene copolymers, and styrene-ethylene/propylene copolymers and combinations thereof. Specifically, rheology enhancers such as mineral oil and ethylene/propylene/styrene copolymers, and mineral oil and butylene/ethylene/styrene copolymers (Versagel blends from Penreco) are particularly preferred. Also, Vistanex (Exxon) and Presperse (Amoco) polymers are particularly suitable rheology enhancers. Other suitable examples of oil-soluble rheology enhancers include, but are not limited to, aluminum stearate, aluminum tristearate, arachidyl alcohol, arachidyl behenate, behenyl alcohol, C₈₋₂₂ alkyl acrylate/butyl dimethicone methacrylate copolymer, C₁₂₋₂₂ alkyl acrylate/hydroxyethylacrylate copolymer, C₁₈₋₃₈ alkyl, C₂₄₋₅₄ acid ester, C₂₀₋₂₄ alkyl dimethicone, C₂₄₋₂₈ alkyl dimethicone, C₃₀₋₆₀ alkyl dimethicone ceresin, cerotic acid, cetearyl alcohol, cetearyl dimethicone/vinyl dimethicone crosspolymer, cetyl alcohol, cetyl glycol, dibehenyl fumarate, hydrogenated polyisobutene, hydrogenated oils, isocetyl alcohol, isocetyl stearoyl stearate, is ophthalic acid/pentaerythritol crosspolymer benzoate/isostearate, isostearyl alcohol, isostearyl stearoyl stearate, jojoba alcohol, lanolin alcohol, lanolin wax, neopentyl glycol dicaprate, neopentyl glycol dicaprylate/dicaprate, neopentyl glycol dicaprylate/dipelargonate/dicaprate, neopentyl glycol diethylhexanoate, neopentyl glycol diheptanoate, neopentyl glycol diisostearate, neopentyl glycol dilaurate, ozokerite, palm alcohol, palm kernel alcohol, paraffin, pentaerythrityl tetramyristate, pentaerythrityl tetraoleate, pentaerythrityl tetrapelargonate, pentaerythrityl tetrastearate, pentaerythrityl trioleate, silica, synthetic beeswax, synthetic candelilla wax, synthetic carnauba, vinyldimethyl/trimethylsiloxysilicate, stearyl dimethicone crosspolymer VP/eicosene copolymer and VP/hexadecene copolymer. Water soluble or water dispersable rheology modifiers include, but are not limited to, acetamide MEA, acrylamide/ethalkonium chloride acrylate Copolymer, acrylamide/ethyltrimonium chloride acrylate/ethalkonium chloride acrylate copolymer, acrylamides copolymer, acrylamide/sodium acrylate copolymer, acrylates/acetoacetoxyethyl methacrylate copolymer, acrylates/beheneth-25 methacrylate copolymer, acrylates/C₁₀₋₃₀ alkyl acrylate crosspolymer, acrylates/ceteth-20 itaconate copolymer, acrylates/ceteth-20 methacrylate copolymer, acrylates/laureth-25 methacrylate copolymer, acrylates/palmeth-25 acrylate copolymer, acrylates/palmeth-25 itaconate copolymer, acrylates/steareth-50 acrylate copolymer, acrylates/steareth-20 itaconate copolymer, acrylates/steareth-20 methacrylate copolymer, acrylates/stearyl methacrylate copolymer, acrylates/vinyl isodecanoate crosspolymer, acrylates/vinyl neodecanoate crosspolymer, acrylic acid/acrylonitrogens copolymer, agar, agarose, algin, alginic acid, ammonium acryloyldimethyltaurate/vinyl formamide copolymer, ammonium acryloyldimethyltaurate/VP copolymer, ammonium alginate, ammonium chloride, amylopectin, avena sativa (oat) kernel flour, bentonite, calcium alginate, calcium carrageenan, C₂₀₋₄₀ alkyl stearate, carbomer, carboxybutyl chitosan, carboxymethyl hydroxyethylcellulose, carboxymethyl hydroxypropyl guar, cassia Gum, cellulose gum, cetyl hydroxyethylcellulose, C₁₂₋₁₄ hydroxyalkyl, hydroxyethyl sarcosine, cocamide DEA, cocamide MEA, decyl HDI/PEG-180 crosspolymer, decyltetradeceth-200 isostearate, dextrin, dimethicone/PEG-10 crosspolymer, dimethicone/PEG-15 crosspolymer, dimethylacrylamide/ethyltrimonium chloride methacrylate copolymer, disteareth-75 IPDI, disteareth-100 IPDI, gelatin, gellan gum, hectorite, hydrated silica, hydrolyzed cellulose gum, hydroxybutyl methylcellulose, hydroxyethylcellulose, hydroxyethyl ethylcellulose, hydroxypropyl chitosan, hydroxypropyl guar, hydroxypropyl methylcellulose, hydroxypropyl methylcellulose stearoxy ether, hydroxypropyl starch, hydroxypropyl starch phosphate, hydroxypropyl xanthan gum, isopolyglyceryl-3 dimethicone, isopolyglyceryl-3 dimethiconol, lauryl hydroxysultaine, lauryl/myristyl glycol hydroxypropyl ether, lauryl PEG-9 polydimethylsiloxyethyl dimethicone, lauryl polyglyceryl-3 polydimethylsiloxyethyl dimethicone, levan, magnesium alginate, magnesium aluminum silicate, magnesium silicate, magnesium trisilicate, methoxy PEG-22/dodecyl glycol copolymer, methylcellulose, methyl ethylcellulose, methyl hydroxyethylcellulose, microcrystalline cellulose, montmorillonite, myristamidopropyl hydroxysultaine, oatamidopropyl betaine, octacosanyl glycol isostearate, octadecene/MA copolymer, pectin, PEG-150/decyl alcohol/SMDI copolymer, PEG-175 diisostearate, PEG-190 distearate, PEG-15 glyceryl tristearate, PEG-140 glyceryl tristearate, PEG-240/HDI copolymer bis-decyltetradeceth-20 ether, PEG-100/IPDI copolymer, PEG-180/laureth-50/TMMG copolymer, PEG-10/lauryl dimethicone crosspolymer, PEG-15/lauryl dimethicone crosspolymer, PEG-2M, PEG-5M, PEG-7M, PEG-9M, PEG-14 MPEG-20M, PEG-23M, PEG-25M, PEG-45M, PEG-65M, PEG-90M, PEG-115M, PEG-160M, PEG-180M, PEG-120 methyl glucose triisostearate, PEG-120 methyl glucose trioleate, PEG-150 pentaerythrityl tetrastearate, PEG/PPG-120/10 trimethylolpropane trioleate, PEG/PPG-120/10 trimethylpropane trioleatePEG-150/stearyl alcohol/SMDI copolymer, polyacrylate-3, polyacrylate-10, polyacrylate-11, polyacrylic acid, polycyclopentadiene, polyester-5, polyether-1, polyethylene/isopropyl maleate/MA copolyol, polyglycerin-20, polyglycerin-40, polyglyceryl-3 disiloxane dimethicone polyglyceryl-3 polydimethylsiloxyethyl dimethicone, polyquaternium-86, polyvinyl alcohol, potassium polyacrylate, potato starch modified, PVP montmorillonite, sodium acrylates/acrylonitrogens copolymer, sodium acrylates copolymer, sodium acrylates crosspolymer, sodium acrylate/sodium acrylamidomethylpropane sulfonate copolymer, sodium acrylate/sodium acryloyldimethyl taurate/acrylamide copolymer, sodium acrylates/vinyl isodecanoate crosspolymer, sodium acrylate/vinyl alcohol copolymer, sodium acryloyldimethyl taurate/acrylamide/VP copolymer, sodium carboxymethyl beta-glucan, sodium carboxymethyl starch, sodium carrageenan, sodium cellulose sulfate, sodium chloride, sodium hydroxypropyl starch phosphate, sodium isooctylene/MA copolymer, sodium polyacrylate, sodium silicoaluminate, sodium starch octenylsuccinate, sodium sulfate, steareth-100/PEG-136/HDI copolymer, tapioca starch, TEA-alginate, TEA-carbomer, trehalose hydroxypropyltrimonium chloride, tridecyl alcohol, undecyl alcohol, wheat germamidopropyl betaine, xanthan gum, yeast, polysaccharides, and Zea Mays (corn) starch.

Vitamins for use in the adhesive composition include those vitamins commonly used in the skin care industry. Examples include tocopheryl acetate, retinyl palmitate, panthenol, ascorbic acid, and the like, and combinations thereof.

Still other optional components that may be desirable for use in the adhesive composition include those cosmetic and pharmaceutical ingredients commonly used in the skin care industry. Examples include abrasives, absorbents, aesthetic components (fragrances, pigments, colorings/colorants), essential oils, skin sensates, astringents (e.g., clove oil, menthol, camphor, eucalyptus oil, eugenol, menthyl lactate, witch hazel distillate), anti-acne agents, anti-caking agents, antifoaming agents, antioxidants, binders, biological additives, buffering agents, bulking agents, chelating agents, chemical additives, colorants, cosmetic astringents, cosmetic biocides, denaturants, drug astringents, external analgesics, opacifying agents, propellants, reducing agents, sequestrants, skin bleaching and lightening agents (e.g., hydroquinone, kojic acid, ascorbic acid, magnesium ascorbyl phosphate, ascorbyl glucosamine), skin-moisturizing agents, skin-conditioning agents, skin soothing and/or healing agents (e.g., panthenol and derivatives thereof, aloe vera, pantothenic acid and derivatives thereof, allantoin, bisabolol, dipotassium glycyrrhizinate), hemorrhoid treating agents (e.g., witch hazel), prebiotics, probiotics, urinary tract infection treating agents, yeast infection treating agents, bacterial vaginosis treating agents, skin treating agents, sunscreens, thickeners, and vitamins, and combinations thereof. Examples of these and other agents are disclosed in The CTFA Cosmetic Ingredient Handbook, 12^{th} Ed. (2007), which is hereby incorporated by reference to the extent that it is consistent herewith.

The amounts of the optional components will depend on the type of adhesive composition used, as well as the desired benefits of the optional components. In one embodiment, the adhesive composition includes from about 0.001% (by total weight composition) to about 60% (by total weight composition) skin benefit agent and, in another embodiment, from about 0.01% (by total weight composition) to about 30% (by total weight composition) skin benefit agent, and even from about 0.01% (by total weight composition) to about 20% (by total weight composition) skin benefit agent.

More than one skin benefit agent can be used depending on the desired effect(s) of the benefit agents.

The skin benefit agent may be homogeneously dispersed throughout the adhesive composition or may be located at discreet portions of the adhesive composition. For example, a skin benefit agent such as a humectant that functions as a skin moisturizer may be located at the peripheral portions of the adhesive composition that are more likely to contact the skin. Further, additives relating to skin ecology such as prebiotics, probiotics, pH balance agents, bacterial and/or viral infection treating agents and additives could be located at the central portions of the adhesive composition to ensure contact with the skin imperfection. Alternatively or additionally, the skin benefit agent may be micro-embossed or printed on the surface of the adhesive composition and/or first face of the substrate of the adhesive patch. In some embodiments, the skin benefit agent is microencapsulated and dispersed throughout the adhesive composition.

Substrate 200 further includes a second face 215 on the opposite side from the first face 210. During application of the adhesive patch 205, a user's finger will contact the second opposing face 215, also referred to herein as the outer-facing surface, to manipulate adhesive patch 205 onto the skin imperfection.

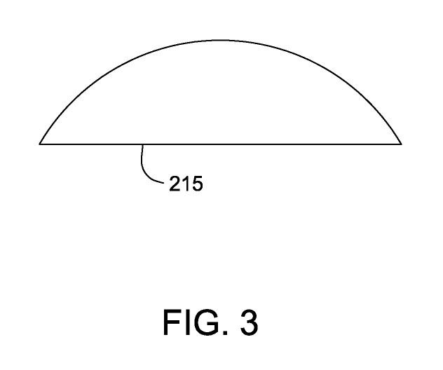

In some embodiments, it may be advantageous for the second opposing face to have a three-dimensional shape. More particularly, the second opposing face 215 may be embossed, printed or otherwise imparted with a pattern to allow easy handling, application and removal of the adhesive patch 205 from the skin imperfection. In one particular embodiment, as shown in FIG. 3, the second opposing face is convex, or outwardly rounded, in shape. Other shapes and textures may include, for example, rigid lines, raised shapes (e.g., circles, triangles, ovals, and the like), and the like, and combinations thereof.

In some embodiments, to further provide ease of handling of the adhesive patches, the second opposing face 215 may include a second adhesive composition (not shown). The second adhesive composition removably adheres the second opposing face of the adhesive patch to a user's finger to aid in applying the adhesive patch to the skin imperfection. The second adhesive composition is also a pressure sensitive adhesive composition similar to the adhesive composition described for use with the first face above.

When a second adhesive composition is applied to the second opposing face, it is desirable that the first adhesive composition applied to the first face have a greater peel strength relating to the attachment between the adhesive composition and the user's skin (i.e., skin imperfection) than the peel strength relating to the attachment between the second adhesive composition and the user's skin (i.e., user's finger) so as to allow the adhesive patch to be easily removed from the user's finger and applied to the skin imperfection. For example, it may be desirable for the first adhesive composition applied to the first face to have a peel strength of composition to skin of from about 5 grams per inch to about 100 grams per inch, including from about 20 grams per inch to about 100 grams per inch, and including from about 50 grams per inch to about 70 grams per inch, and for the second adhesive composition applied to the second opposing face to have a peel strength of composition to user's finger of less than about 50 grams per inch, less than about 25 grams per inch, less than about 20 grams per inch or even less than about 5 grams per inch.

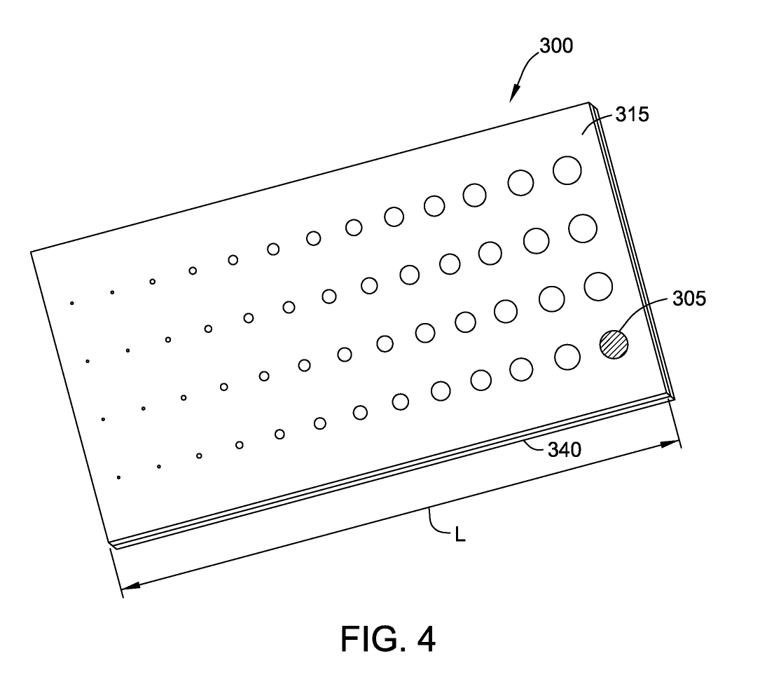

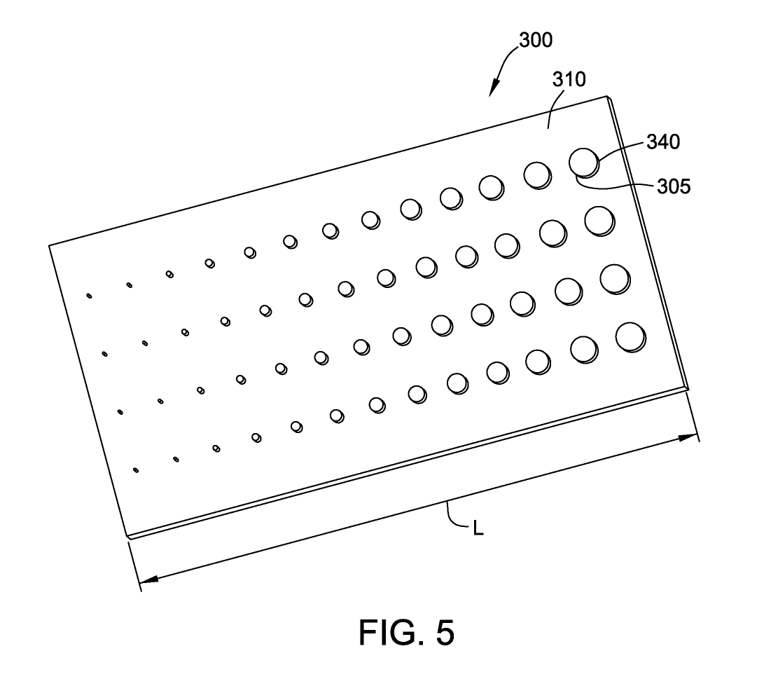

As is shown in FIG. 4, strip 300 with second face 315 facing the viewer. A single adhesive patch 305 has been removed. In this embodiment, to protect the adhesive composition of the first face (not shown), a peel sheet or release sheet 340 may be used to prevent the adhesive composition from becoming contaminated, thus losing its ability to stick to the skin of the user and/or prematurely adhering to an unintended surface. Suitable materials for use as a peel strip 340 are well known in the art and are commercially available. Examples of suitable peel sheets or release sheets include, a silicone coated Kraft paper, a silicone coated film or the like. Other peel sheets include coating containing polytetrafluoroethylene. The peel sheet or release sheet 340 may extend beyond one or both of the ends and/or sides of the adhesive patch, as shown in FIG. 4. Alternatively, the peel sheet 340 may be sized and configured to only cover the adhesive composition on the first face of the substrate, as is shown in FIG. 5. Particularly, as shown in FIG. 5, a strip 300 is shown with the first face 310 facing the viewer and where a peel sheet 340 is being removed from the first face of adhesive patch 305.

In another alternative, a peel sheet may not be necessary. For example, the strips may be rolled, folded onto itself or stacked upon each other. In these configurations, a peel sheet is not needed. In one possible configuration, the strip 300 may be folded along the longitudinal axis L of the lateral axis such that the adhesive composition 340 in one area comes into contact with adhesive composition in another area. In the folded configuration, the adhesive composition should be selected such that the adhesive composition will release from itself when manipulated by a user.

### Methods of Use

In use, the adhesive patches of the present disclosure are directly applied to a skin imperfection such to protect the skin imperfection from a sunless tanning product. More particularly, the methods include identifying a skin imperfection on the skin of a user; applying an adhesive patch to the skin imperfection; applying a sunless tanning product; and removing the adhesive patch from the skin imperfection.

The methods of the present disclosure allow for the protection of skin imperfections from discoloration and/or darkening by sunless tanning products. In many embodiments, the methods may be used to protect skin imperfections such as, for example, age spots, freckles, acne, wounds, scars, birthmarks, chloasma patches, actinic keratosis, actinic lentigines, rashes, and hives. Thus, the methods may be used to minimize/reduce/eliminate the exposure of the skin imperfections of an individual to the coloring effects of the sunless tanning agents typically incorporated into the sunless tanning products. As such, in some embodiments, the methods disclosed herein are directed to a subset of the general population, including individuals with skin imperfections, such that, in these embodiments, not all of the general population may benefit from the methods. Based on the foregoing, because some of the method embodiments of the present disclosure are directed to specific subsets or subclasses of identified individuals (that is, the subset or subclass of individuals “in need” of assistance in addressing discoloration and/or darkening of skin imperfections during sunless tanning), not all individuals will fall within the subset or subclass of individuals as described herein.

Once a skin imperfection is identified, an adhesive patch is applied to the skin imperfection using any means known in the art. Typically, the adhesive patch is applied by removing an adhesive patch from a strip with the user's finger and applying the patch to the skin imperfection by pressing the patch to the skin imperfection to activate the pressure sensitive adhesive composition on the first face of the substrate of the adhesive patch, thereby adhering the patch to the skin imperfection.

The method further includes applying a sunless tanning product to the skin of the user. The sunless tanning product typically includes a sunless tanning agent to color the user's skin to provide the appearance of a tan such as is produced during exposure of the skin to sun. Typically, the sunless tanning product is applied for a time sufficient to provide uniform desired color development. Times will typically depend on the user's skin tone, as well as on the sunless tanning agent employed in the product and the other compositions of the sunless tanning product. Generally, the sunless tanning product is applied for a period of at least 1 minute, including at least 5 minutes, including at least 10 minutes, including at least 15 minutes, including at least 20 or longer. In some embodiments, the sunless tanning product is applied for a period of from about 1 minute to about 30 minutes, including from about 1 minute to about 25 minutes, and including from about 1 minute to about 20 minutes.

The sunless tanning product may be applied to the user's skin in any form as known in the art, including, for example, in the form of a lotion, gel, cream, aerosol spray, non-aerosol spray, sol, and combinations thereof.

After a time period to allow for sufficient, uniform color development, application of the sunless tanning product is ceased and the adhesive patch is removed by peeling the patch from the skin imperfection.

Advantageously, the adhesive patches and methods of the present disclosure provide for a more uniformly tanned appearance of the user's skin. Further, in protecting the skin imperfection, which is typically different in appearance (e.g., color, texture, etc.) than the skin surrounding the imperfection, upon coloring/darkening of the skin with the sunless tanning product, the skin imperfection is less visible as the imperfection and surrounding skin will have similar coloring.

In view of the above, it will be seen that the several advantages of the disclosure are achieved and other advantageous results attained. As various changes could be made in the above methods and adhesive patches without departing from the scope of the disclosure, it is intended that all matter contained in the above description and shown in the accompanying drawings shall be interpreted as illustrative and not in a limiting sense.

When introducing elements of the present disclosure or the various versions, embodiment(s) or aspects thereof, the articles “a”, “an”, “the” and “said” are intended to mean that there are one or more of the elements. The terms “comprising”, “including” and “having” are intended to be inclusive and mean that there may be additional elements other than the listed elements.

What is claimed is:

1. An adhesive patch for protecting skin imperfections during sunless tanning, the adhesive patch comprising: a substrate and an adhesive composition on at least a first face of the substrate, the substrate having a width along its greatest extent of from about 1/16″ to about 7/16″.

2. The adhesive patch as set forth in claim 1, wherein the substrate comprises a material selected from the group consisting of woven materials, nonwoven materials, composite materials, polymeric films, film-nonwoven fabric laminates, and combinations thereof.

3. The adhesive patch as set forth in claim 1, wherein the substrate is porous.

4. The adhesive patch as set forth in claim 1, wherein the substrate comprises a second face on an opposing side from the first face, the second face having a three-dimensional shape.

5. The adhesive patch as set forth in claim 4, wherein the three-dimensional shape comprises a convex shape.

6. The adhesive patch as set forth in claim 1, wherein the adhesive composition is a pressure sensitive adhesive composition selected from the group consisting of hydrogels, hydrocolloids, acrylics-based adhesives, rubber-based adhesives, and combinations thereof.

7. The adhesive patch as set forth in claim 1, wherein the adhesive composition is a first adhesive composition and the adhesive patch further comprises a second adhesive composition on a second opposing face of the substrate, the first adhesive composition has an adhesive peel strength greater than the adhesive peel strength of the second adhesive composition.

8. The adhesive patch as set forth in claim 7 further comprising a peel sheet in contact with the first face of the substrate.

9. The adhesive patch as set forth in claim 1, wherein the skin imperfection is selected from the group consisting of age spots, wounds, scars, acne, and combinations thereof.

10. The adhesive patch as set forth in claim 1, wherein the adhesive composition comprises one or more skin benefit agents selected from the group consisting of antiperspirants, deodorants, skin moisturizers, humectants, pH modulators, soothing agents, antimicrobials, preservatives, film formers, abrasives, absorbents, aesthetic components, essential oils, skin sensates, astringents, anti-acne agents, anti-caking agents, antifoaming agents, antioxidants, binders, biological additives, buffering agents, bulking agents, chelating agents, chemical additives, colorants, cosmetic astringents, cosmetic biocides, denaturants, drug astringents, external analgesics, opacifying agents, propellants, reducing agents, sequestrants, skin treating agents, sunscreens, thickeners, vitamins, and combinations thereof.

11. A strip comprising a plurality of adhesive patches for protecting skin imperfections during sunless tanning, the plurality of adhesive patches comprising at least a first adhesive patch and a second adhesive patch, the first adhesive patch and the second adhesive patch independently comprising a substrate and an adhesive composition on at least a first face of the substrate, wherein the first adhesive patch and the second adhesive patch independently have a width along their greatest extents of from about 1/16″ to about 7/16″, and wherein the width along its greatest extent of the first adhesive patch is greater than the width along its greatest extent of the second adhesive patch.

12. The strip as set forth in claim 11, wherein the plurality of adhesive patches are arranged in columns, rows, or combinations thereof.

13. The strip as set forth in claim 11, wherein the substrate of at least one of the first adhesive patch and second adhesive patch is porous.

14. The strip as set forth in claim 11, wherein the substrate of at least one of the first adhesive patch and second adhesive patch comprises a second face on an opposing side from the first face, the second face having a three-dimensional shape.

15. The strip as set forth in claim 11, wherein the adhesive composition is a first adhesive composition and at least one of the first adhesive patch and second adhesive patch further comprises a second adhesive composition on a second opposing face of the substrate, the first adhesive composition has an adhesive peel strength greater than the adhesive peel strength of the second adhesive composition.

16. The strip as set forth in claim 15 further comprising a peel sheet in contact with the first face of the substrate.

17. The strip as set forth in claim 11, wherein the adhesive composition comprises one or more skin benefit agents selected from the group consisting of antiperspirants, deodorants, skin moisturizers, humectants, pH modulators, soothing agents, antimicrobials, preservatives, film formers, abrasives, absorbents, aesthetic components, essential oils, skin sensates, astringents, anti-acne agents, anti-caking agents, antifoaming agents, antioxidants, binders, biological additives, buffering agents, bulking agents, chelating agents, chemical additives, colorants, cosmetic astringents, cosmetic biocides, denaturants, drug astringents, external analgesics, opacifying agents, propellants, reducing agents, sequestrants, skin treating agents, sunscreens, thickeners, vitamins, and combinations thereof.

18. A method of protecting skin imperfections during sunless tanning, the method comprising:
identifying a skin imperfection on the skin of a user;
applying an adhesive patch to the skin imperfection;
applying a sunless tanning product; and
removing the adhesive patch from the skin imperfection.

19. The method as set forth in claim 18, wherein the sunless tanning product is applied for a period of from about 1 minute to about 20 minutes.

20. The method as set forth in claim 18, wherein the sunless tanning product is applied in a form selected from the group consisting of lotion, gel, cream, aerosol spray, non-aerosol spray, sol, and combinations thereof.
